(19) United States
(12) Patent Application Publication
(10) Pub. No.: US 2008/0287752 A1
(43) Pub. Date: Nov. 20, 2008

(54) EAR CANAL PHYSIOLOGICAL PARAMETER MONITORING SYSTEM

(75) Inventors: Randolph W. Stroetz, Rochester, MN (US); Bruce J. Walters, Mazeppa, MN (US); Steven R. Holets, Oronoco, MN (US); Jan Stepanek, Scottsdale, AZ (US); Thomas E. Belda, Rochester, MN (US); Curtis F. Buck, Plainview, MN (US)

Correspondence Address: FAEGRE & BENSON;ATTN: PATENT DOCKETING, 2200 WELLS FARGO CENTER, 90 SOUTH SEVENTH STREET, MINNEAPOLIS, MN, 55402-3901, US

(73) Assignee: Mayo Foundation for Medical Education and Research, Rochester, MN (US)

(21) Appl. No.: 12/117,758

(22) Filed: May 9, 2008

Related U.S. Application Data
(60) Provisional application No. 60/928,558, filed on May 10, 2007.

(51) Int. Cl. A61B 5/02; A61B 5/12

(52) U.S. Cl. 600/301; 600/559

(57) ABSTRACT

Physiological parameters are monitored from within an ear canal. A sensor module, which is mountable within the ear canal, includes at least one sensor operable to sense the physiological parameters and to generate signals based on the sensed physiological parameters. A processor, which is in communication with the sensor module, processes the signals from the at least one sensor and generates data related to the physiological parameters.

### CROSS-REFERENCE TO RELATED APPLICATION(S)

The present application claims the benefit of U.S. Provisional Application No. 60/928,558 filed May 10, 2007, entitled “Ear Canal Physiologic Parameter Monitoring System,” which is incorporated herein by reference in its entirety.

### FIELD

The present invention relates to measuring physiological parameters. More particularly, the present invention relates to a system for measuring and monitoring physiological parameters from within an ear canal.

### BACKGROUND

The frequent measurement and monitoring of certain physiological parameters is not only desirable for certain groups of people, but also critical to their health, performance, and well-being. For example, some military personnel may be subjected to extreme environmental conditions or periods of high gravitational loading that can lead to adverse medical conditions such as hypoxia, hypocapnia, hypothermia, hyperthermia, or gravitational loss of consciousness. As another example, vulnerable individuals, people with chronic medical conditions, and the elderly may have a propensity for certain ailments or a higher likelihood of suffering a falling incident. Physiological parameters for these and other groups of people should be monitored constantly or near-constantly to assure that appropriate measures can be taken upon the occurrence of an adverse medical event.

Monitoring of physiological parameters involves the measurement and analysis of certain characteristics of the body. For example, a pulse oximetry test may be used to measure the level of oxygen saturation in the blood, which can be used to establish whether conditions such as hypoxia exist. To provide constant monitoring of these physiological parameters, sensors may be attached to portions of the exterior of the body to provide data relevant to the physiological parameter being measured. However, connection of these sensors to exterior surfaces of the body leaves them vulnerable to inadvertent damage that can lead to failure of the sensors. In addition, environmental conditions, such as ambient light or sound, can affect the measurements of an externally mounted sensor. This becomes a particularly important consideration when even small variations in a physiological parameter can cause deleterious effects on human performance.

### SUMMARY

The present invention relates to monitoring one or more physiological parameters from within an ear canal. A sensor module, which is mountable within the ear canal, includes at least one sensor that senses the one or more physiological parameters and generates signals based on the sensed physiological parameters. A processor, which is in communication with the sensor module, processes the signals from the at least one sensor and generates data related to the one or more physiological parameters.

### BRIEF DESCRIPTION OF THE DRAWINGS

FIG. 1 is a diagrammatic and block diagram of a sensor module secured in an ear canal and connected to a processor that communicates with a remote location in accordance with an embodiment of the invention.

FIG. 2 is a block diagram of the sensor module shown in FIG. 1, including a plurality of physiological sensors.

FIG. 3 is a flow diagram of a process for employing data and voice communication via the sensor module shown in FIG. 1 in accordance with an embodiment of the invention.

### DETAILED DESCRIPTION

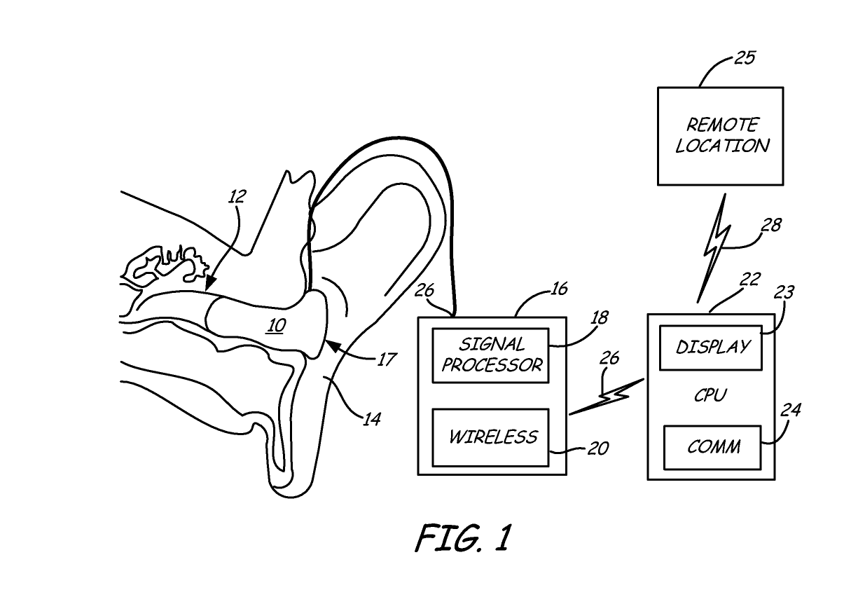

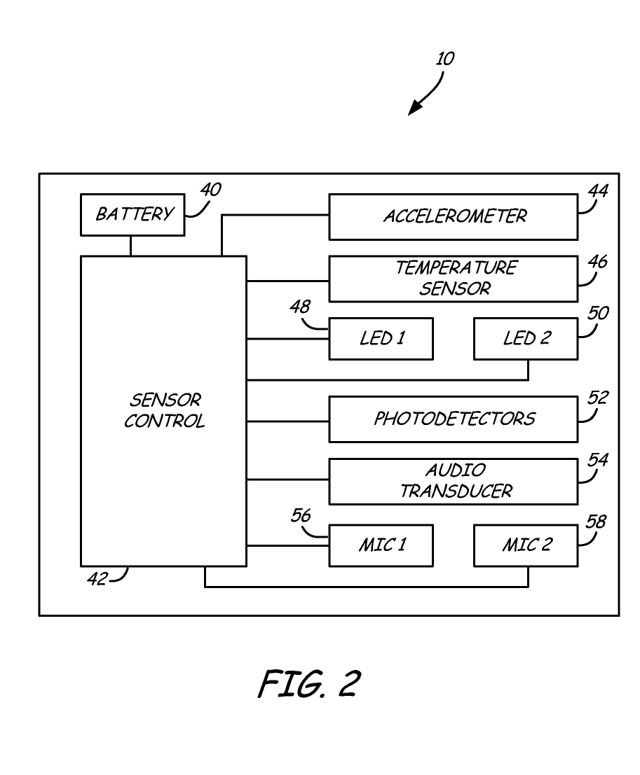

FIG. 1 is a diagrammatic and block diagram view of sensor module 10 secured in ear canal 12 of human ear 14 and connected to sensor processor module 16. Sensor module 10 includes one or more sensors in an ear-canal shaped housing 17 that are positioned relative to portions of ear canal 12 to measure physiological parameters of the user. Examples of sensors that may be included in sensor module 10 will be described in more detail with regard to FIG. 2. Sensor processor module 16 includes signal processor 18 for communicating with and processing signals from sensor module 10. Sensor processor module 16 also includes wireless interface 20 for communicating data and other information from signal processor 16 to central processing unit (CPU) 22. CPU 22, which includes display 23 and communication module 24, communicates with remote location 25 wirelessly.

Sensor module 10 is connected to sensor processor module 16 via transmission line 26. Alternatively, sensor module 10 and sensor processor module 16 may communicate with each other via a wireless connection. Sensor processor module 16 is carried by the user in close proximity (e.g., within one to two meters) to sensor module 10. Sensor processor module 16 is typically carried or secured on the user's body. For example, sensor processor module 16 may be secured behind the flap or pinna of ear 14, or sensor processor module 16 may be configured for placement in the user's pocket.

Sensor processor module 16 receives and processes signals related to physiological parameters that are measured by sensors in sensor module 10. Signal processor 18 may include various signal processing elements to manipulate the signals from sensor module 10 for subsequent processing and analysis. For example, signal processor 18 may include an analog-to-digital (A/D) converter, a digital signal processor (DSP), signal filters, and/or a signal conditioner.

When the signals from sensor module 10 have been processed by signal processor 18, sensor processor module 16 transmits the signals wirelessly to CPU 22 via wireless interface 20. In some embodiments, wireless connection 26 is a low-power, close-range wireless protocol, such as Bluetooth, to limit the level of electromagnetic radiation that is transmitted around the user's head. In an alternative embodiment, sensor processor module 16 communicates with CPU 22 via a wired connection, such as through a USB cable. CPU 22 may be housed in an assembly that is adapted to be worn locally by the user, such as on the user's belt. In an alternative embodiment, CPU 22 is provided in an assembly located remotely from the user, such as a personal computer. In another alternative embodiment, CPU 22 is integrated with sensor processor module 16. In a further alternative embodiment, sensor processor module 16 and CPU 22 are integrated into sensor module 10.

When CPU 22 receives signals from sensor processor module 16, CPU 22 analyzes the signals to generate data related to the measured physiological parameters. In some embodiments, CPU 22 runs the signals through one or more monitoring algorithms to generate relevant data. For example, CPU 22 may generate data related to the breathing rate and breath sounds of the user by analyzing acoustic signals from sensor module 10. As another example, CPU 22 may analyze optical data related to pulse oximetry measurements by sensor module 10 to generate data related to the level of oxygen saturation in the blood. CPU 22 may include algorithms to analyze any physiological signals measurable from within ear canal 12. In addition to those described above, these signals can be used to monitor physiological parameters including, but not limited to, core body temperature, heart rate, carboxyhemoglobin levels, transcranial Doppler signals, inspiratory to expiratory (I/E) ratio, sound of blood flow in the carotid artery, an index of perfusion status at head level.

The data related to the measured physiological parameters generated by CPU 22 may then be output for review or monitoring. For example, CPU 22 may provide information about the physiological parameters and system status on display 23 such that the user can monitor the measured physiological parameters locally. CPU 22 may also transmit the data to remote location 25 with communication module 24 through wireless connection 28, such as a wireless fidelity (WiFi) or satellite connection. Alternatively, CPU 22 may communicate with remote location 25 via a wired connection.

Remote location 25 is a facility that monitors the physiological data provided by CPU 22 and provides an appropriate response when the data indicates certain (e.g., deteriorating) physiological conditions. For example, when the physiological parameters of military personnel are being monitored, such as during exposure to extreme conditions, remote location 25 may be a medical facility on a military base. As another example, when physiological parameters of vulnerable, chronically ill, or elderly patients are being monitored, remote location 25 may be a primary care facility or emergency response center. The response provided by remote location 25 is based on the type of monitoring being conducted on the user, and may include data collection, interaction with the user, and/or dispatch of emergency personnel. CPU 22 is also operable to receive electronic communications from remote location 25 via communication module 24. CPU 22 may also provide information or alerts to the user and/or contact emergency personnel directly when the data indicates certain physiological conditions.

FIG. 2 is a block diagram of an embodiment of sensor module 10, which includes battery 40, sensor controller 42, accelerometer 44, temperature sensor 46, light-emitting diodes 48 and 50, photodetectors 52, audio transducer 54, and microphones 56 and 58. Battery 40 provides power to each of the various sensors and sensor controller 42. Accelerometer 44, temperature sensor 46, light-emitting diodes (LEDs) 48 and 50, photodetectors 52, audio transducer 54, and microphones 56 and 58 are each connected to sensor controller 42. It should be noted that the sensors shown are merely by way of example, and it will be appreciated that any types of sensors capable of measuring physiological parameters from within ear canal 12 may also be incorporated into sensor module 10.

Sensor controller 42 is operable to provide control signals to and receive measurement signals from sensors in sensor module 10. Sensor controller 42 provides sensor excitation signals to the sensors when a measurement is to be conducted by that sensor, and amplifies the signals provided by the sensors after a measurement. Sensor controller 42 also controls the power that is provided to LEDs 48 and 50 during pulse oximetry measurements. Sensor controller 42 then transmits the sensed physiological signals generated by the sensor to sensor processor module 16.

Accelerometer 44 is a linear accelerometer that measures the acceleration of the user's body along any of up to the three spatial axes. Signals from accelerometer 44 may be analyzed by CPU 22 to establish when the user is being subjected to high gravitational forces, such as those a military pilot may be subjected to during flight. The signals generated by accelerometer 44 may also be analyzed by CPU 22 to determine when a user has had a falling incident. For example, an acceleration threshold may be programmed into CPU 22 that, when reached or exceeded, indicates a likelihood that a falling incident has occurred.

Temperature sensor 46 measures the core body temperature of the user from within ear canal 12. In some embodiments, temperature sensor 46 is mounted in sensor module 10 to measure the temperature at the tympanic membrane of ear canal 12.

LEDs 48 and 50 operate in conjunction with photodetectors 52 to provide signals related to pulse oximetry measurements. In some embodiments, LED 48 emits red light and LED 50 emits infrared light. Photodetectors 52 are positioned in sensor module 10 to detect optical signals after light from LEDs 48 and 50 passes through tissue. For example, LEDs 48 and 50 and photodetectors 52 may be positioned on opposite sides of the tragus of ear 14. The changing absorbance across the tissue of each of the wavelengths of LEDs 48 and 50 is measured by photodetectors 52, which allows for a determination of the oxygen saturation in the blood.

Audio transducer 54 is positioned in sensor module 10 to provide audible signals to the user. For example, audio transducer 54 may emit an alert tone to a user in response to a change in a physiological parameter. In some embodiments, audio transducer 54 is positioned proximate the innermost portion of sensor module 10 in ear canal 12. This arrangement provides a direct path to deliver the sound from audio transducer 54 to the tympanic membrane.

Microphones 56 and 58 are employed to receive acoustic signals to measure physiological parameters having audio characteristics. Microphones 56 and 58 may be arranged in sensor module 10 such that one of microphones 56 and 58 measures physiologic parameters within ear canal 12 while the other of microphones 56 and 58 measures sound external to ear canal 12. When microphones 56 and 58 are arranged in this way, a noise cancellation algorithm may be employed by CPU 22 to subtract the noise external to ear canal 12 from the acoustic measurement from within ear canal 12. As a result, physiological sounds can be isolated from other ambient sounds. In addition, the arrangement of microphones 56 and 58 may be used to differentiate speech and sounds of the user from other external sounds.

The combination of audio transducer 54 and microphones 56 and 58 may also function as a hearing aid for the user. The microphone 56 or 58 that is arranged to measure sound external to ear canal 12 receives sounds from people and other external sources and provides signals to CPU 22. CPU 22 then converts the signals for use by audio transducer 54, which amplifies the signal for delivery to the tympanic membrane. A volume control interface may be provided on sensor processor module 16 or CPU 22 to allow the user to control the level of audio amplification provided to the tympanic membrane. CPU 22 may also employ an algorithm to separate voice sounds from other extraneous noise.

In certain cases, the physiological parameters of the user are constantly monitored by remote location 25 to assure the continued health and well-being of the user. CPU 22 may employ a threshold based algorithm to establish whether a physiological parameter remains within parameter limits. In some embodiments, CPU 22 employs a threshold based decision tree algorithm. Parameter thresholds may be programmed into CPU 22 that, when reached or exceeded, indicate that a likelihood that an emergency medical incident is occurring or has occurred. For example, a threshold oxygen saturation level may be programmed into CPU 22 that, when reached or exceeded, indicates the user is likely suffering from hypoxia.

CPU 22 may be also programmed to establish a communication link with remote location 25 when a physiological parameter threshold is reached or exceeded. This may be a data connection that provides information related to the physiological parameter of concern to remote location 25. Personnel at remote location 25 may then take appropriate response measures, such as dispatching of emergency personnel to the user's location. Alternatively, response measures may be automatically taken at remote location 25 when the data connection between CPU 22 and remote location 25 is established due to the physiological parameter threshold being reached or exceeded.

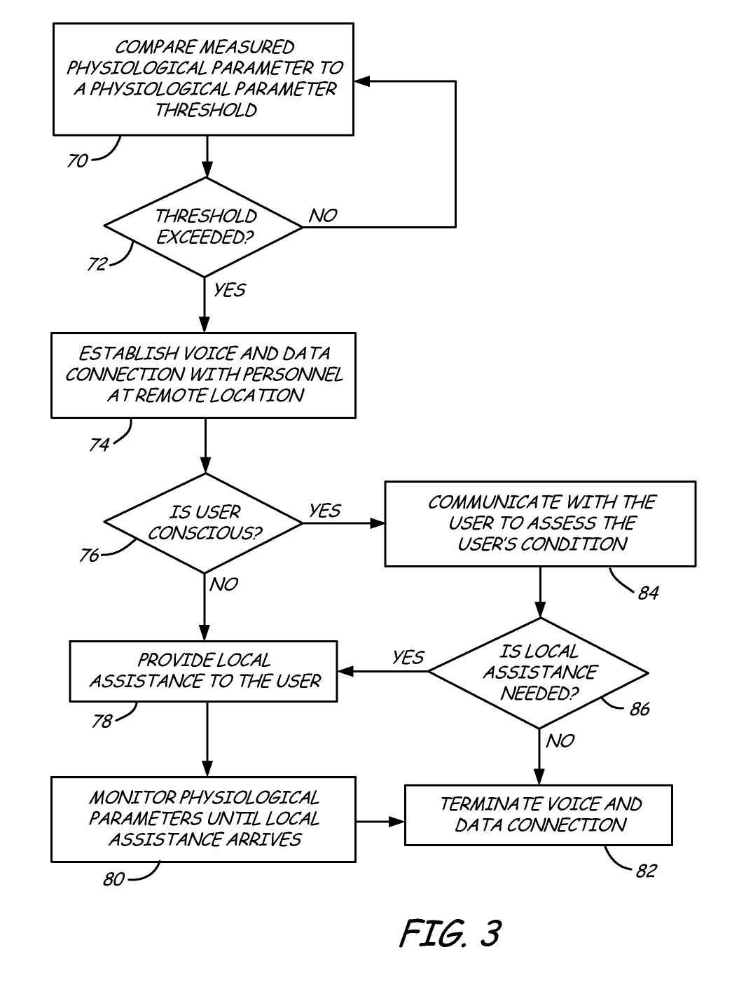

FIG. 3 is a flow diagram of a process for establishing a voice and data connection between sensor module 10 and remote location 25 based on the monitored physiological parameters, according to another embodiment of the present invention. In step 70, each of the physiological parameters measured by sensor module 10 is compared with a corresponding physiological parameter threshold. The comparison may be performed by any of signal processor module 16, CPU 22, or sensor control module 42.

In step 72, if the physiological parameter threshold is not exceeded for any of the monitored physiological parameters, the process returns to step 70. If any of the measured physiological parameters exceeds its corresponding physiological parameter threshold in step 72, then a voice and data connection is established with personnel at remote location 25 in step 74. For example, CPU 22 may establish a data and voice over internet protocol (VoIP) connection through communication module 24. Examples of events that may trigger step 74 include, but are not limited to, a fall by the user, a lack of movement by the user for a threshold period of time, high or low heart rate, low blood oxygen level, and/or high respiratory rate.

In step 76, personnel at remote location 25 determine whether the user of sensor module 10 is conscious. This determination may be based on certain physiological parameters measured by sensor module 10, such as breathing patterns and pulse rate, that may indicate a likelihood that the user is unconscious. The determination of consciousness may also be made by personnel at remote location 25 orally communicating with the user to establish the severity of the condition. The voice of personnel at remote location 25 is heard by the user via audio transducer 54, and the user's voice and physiological sounds are received by microphones 56 and/or 58 and transmitted to remote location 25. If the user is unable to orally communicate, personnel at remote location 25 can assess pulse rate, breathing patterns, and breath sounds through the signals received by microphones 56 and 58. With the noise-cancelling arrangement of microphones 56 and 58, breathing sounds, as well as moaning and other distressed sounds, are also isolated and transmitted to remote location 25. In this way, personnel at remote location 25 can verify that the audio components of sensor module 10 are functioning properly, even if the user is unable to orally communicate. Personnel at remote location 25 can then assess the user's level of consciousness based on the audio signals transmitted to remote location 25 from sensor module 10.

In step 78, if personnel at remote location 25 determine that the user is unconscious, measures are taken by the personnel to provide local assistance to the user. For example, if the user is an at-home patient whose physiological parameters are being monitored, the personnel may contact an emergency service provider to dispatch assistance locally to the user, such as by sending an ambulance to the patient's residence.

In step 80, personnel at remote location 25 continue to monitor the physiological parameters of the user until assistance local to the user arrives. This allows the personnel to assess the condition of the patient before the local assistance arrives, such as by analyzing blood flow characteristics and breathing patterns and sounds of the user to assess the quality of the airway, breathing, and circulation (ABC) of the user. Personnel at remote location 25 may also continuously update the local assistance provider on the status of the user until the local assistance provider arrives. When the local assistance arrives, the voice and data connection with remote location 25 is terminated in step 82.

If, in step 76, personnel at remote location 25 determine that the user is conscious, the personnel at remote location 25 communicate with the user to assess the user's condition in step 82. The personnel may ask a series of questions to the user to determine the level of cognizance of the user. The personnel may also ask the user to describe what he or she is feeling to make an assessment of the user's condition.

In step 86, if personnel at remote location 25 determine that local assistance is not needed, the voice and data connection between CPU 22 and remote location 25 is terminated (step 82). On the other hand, if personnel at remote location 25 determine that the user requires local assistance, the process proceeds through steps 78, 80, and 82 similarly as described above.

In summary, the present invention relates to monitoring one or more physiological parameters from within an ear canal. A sensor module, which is mountable within the ear canal, includes at least one sensor that senses the one or more physiological parameters and generates signals based on the sensed physiological parameters. A processor, which is in communication with the sensor module, processes the signals from the at least one sensor and generates data related to the one or more physiological parameters. The data may be transmitted to a remote location for monitoring and/or appropriate response measures. A voice and data connection may also be established with the remote location to allow personnel at the remote location to assess the user's level of cognizance and consciousness through audio communication, as well as to provide appropriate response measures. The ear canal provides a protected site for measuring physiological data, which minimizes the effect of external environmental conditions, such as ambient light and sound, on the measurement of the physiological signals. Consequently, even small variations in physiological parameters are detectable by the sensor module.

Although the present invention has been described with reference to preferred embodiments, those skilled in the art will recognize that changes can be made in form and detail without departing from the spirit and scope of the invention.

What is claimed is:

1. A system for monitoring one or more physiological parameters from within an ear canal, the system comprising:
a sensor module securable within the ear canal and including at least one sensor operable to sense the one or more physiological parameters and to generate signals based on the sensed physiological parameters; and
a processor in communication with the sensor module and operable to process the signals from the at least one sensor and to generate data related to the one or more physiological parameters.

2. The system of claim 1, and further comprising:
a communication module operable to transmit the data from the processor to a remote location.

3. The system of claim 2, wherein the communication module is further operable to receive electronic communications from the remote location.

4. The system of claim 2, wherein the processor compares the data related to each physiological parameter to a corresponding parameter threshold.

5. The system of claim 4, wherein the communication module communicates with the remote location when at least one of the one or more physiological parameters reaches the corresponding parameter threshold.

6. The system of claim 1, and further comprising:
an output device in communication with the processor that is operable to generate a sensory output based on the data.

7. The system of claim 6, wherein the output device generates the sensory output when at least one of the one or more physiological parameters reaches a parameter threshold.

8. A system for monitoring physiological parameters, the system comprising:
a plurality of physiological sensors each operable to sense at least one physiological parameter;
an ear module securable within an ear canal that houses the plurality of physiological sensors and positions the plurality of physiological sensors relative to the ear canal to sense the physiological parameters; and
a processor in communication with the plurality of physiological sensors and operable to process the signals from the physiological sensors and to generate data related to the one or more physiological parameters.

9. The system of claim 8, and further comprising:
a communication module operable to transmit the data from the processor to a remote location.

10. The system of claim 9, wherein the communication module is further operable to receive electronic communications from the remote location.

11. The system of claim 9, wherein the processor compares the data related to each physiological parameter to a corresponding parameter threshold.

12. The system of claim 11, wherein the communication module communicates with the remote location when at least one of the one or more physiological parameters reaches the corresponding parameter threshold.

13. The system of claim 12, wherein the ear module further includes an audio assembly, and wherein audio signals are communicated between the ear module and the remote location via the audio assembly when at least one of the one or more physiological parameters reaches the parameter threshold.

14. The system of claim 8, wherein the ear module further includes an audio assembly operable to cancel noise external to the ear canal when measuring physiological parameters having audio characteristics.

15. The system of claim 8, and further comprising:
an output device in communication with the processor that is operable to generate a sensory output based on the data.

16. A method for monitoring physiological parameters, the method comprising:
sensing one or more physiological parameters with the at least one physiological sensor in a sensor module mounted within an ear canal; and
generating data related to the sensed physiological parameters.

17. The method of claim 16, and further comprising:
transmitting the data to a remote location.

18. The method of claim 16, and further comprising:
comparing the data related to each physiological parameter to a corresponding parameter threshold.

19. The method of claim 18, and further comprising:
communicating with a remote location when at least one of the one or more physiological parameters reaches the corresponding parameter threshold.

20. The method of claim 19, wherein the communicating step comprises:
transmitting the data to the remote location; and/or
establishing a telephonic connection with the remote location.
